(19) United States
(12) Patent Application Publication
(10) Pub. No.: US 2026/0207511 A1
(43) Pub. Date: Jul. 23, 2026

(54) MACROMOLECULAR DELIVERY SYSTEM FOR ENHANCED BIOAVAILABILITY OF ACTIVE SUBSTANCES

(71) Applicant: EncapsuPharm LLC, Dover, DE (US)

(72) Inventors: Sergey Sorokin, Bavaro (DO); Igor Verba, Kyiv (UA)

(21) Appl. No.: 19/027,580

(22) Filed: Jan. 17, 2025

(51) Int. Cl. A61K 9/20; A61K 9/00

(52) U.S. Cl. CPC A61K 9/2031; A61K 9/0014; A61K 9/2013

(57) ABSTRACT

An aqueous pharmaceutical composition for enhancing bioavailability of an active substance in a patient includes an active substance surrounded by a solubilizer layer having at least one polyethylene glycol selected from the group of PEG-40 glyceryl trihydroxystearate, PEG-35 glyceryl triricinoleate, PEG-15 hydroxystearate, and mixtures thereof, the solubilizer layer encapsulated by an outer layer comprising a composition selected from the group of: 1) a composition comprising phospholipids and 70%-79% phosphatidylcholine, 2) a composition comprising 94%-100% phosphatidylcholine, and 3) analogs thereof, all of the foregoing being a nanodispersed composition that is mixed with water or an aqueous solution to form a stable aqueous formulation suitable for medical use.

### CROSS REFERENCE TO RELATED APPLICATIONS

Not Applicable.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The claimed subject matter relates to the field of pharmaceutical sciences and drug delivery systems. Specifically, the claimed subject matter relates to macromolecular delivery systems and methods for enhancing the bioavailability of active substances, thereby improving the efficacy of preparations.

### BACKGROUND

The effective delivery of active pharmaceutical ingredients (APIs) remains a significant challenge in the fields of medicine, cosmetics, and veterinary science. Various therapeutic agents, particularly those that are poorly soluble in water, exhibit low bioavailability when administered through conventional routes such as oral or topical application. Poor water solubility limits the absorption of these substances into systemic circulation, thereby reducing their therapeutic efficacy.

Various strategies have been developed to enhance the solubility and bioavailability of poorly water-soluble molecules. These include the use of solubilizers, emulsifiers, lipid-based formulations, nanoparticles, micelles, and liposomes. For instance, polymeric nanoparticles and liposomal systems have been employed to encapsulate active substances, protecting them from degradation and enhancing their absorption in the gastrointestinal tract. Micelle-forming surfactants have also been used to increase the solubility of hydrophobic molecules by encapsulating them within hydrophobic cores.

Despite these advancements, existing delivery systems often face limitations that hinder their effectiveness. Liposomes and some polymeric nanoparticles can be unstable in physiological environments, leading to premature release or degradation of the encapsulated agents. Additionally, these systems may require complex manufacturing processes and the use of large quantities of excipients or additional substances, increasing production costs and the potential for adverse reactions.

Further, a significant obstacle in drug delivery is the neutralization of active substances by the body's immune system. Macrophages and other components of the immune system can recognize and eliminate foreign substances, including drug carriers like nanoparticles and liposomes. This immune response reduces the amount of active substance that reaches the target site, diminishing therapeutic outcomes. Existing methods to evade immune detection, such as surface modification of particles with hydrophilic polymers, have had limited success and can introduce new challenges, such as accelerated blood clearance upon repeated administration.

Therefore, there exists a significant need in the prior art for improved delivery systems that enhance the bioavailability of active substances, particularly those that are poorly water-soluble, while overcoming the challenges of immune system neutralization.

### BRIEF SUMMARY

This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

The disclosed embodiments are directed to an aqueous pharmaceutical composition for enhancing bioavailability of an active substance in a patient, which address the problems with the prior art, namely, the challenges of bioavailability and immune system neutralization. In one embodiment, the aqueous pharmaceutical composition comprises an active substance surrounded by a solubilizer layer comprising at least one polyethylene glycol selected from the group consisting of PEG-40 glyceryl trihydroxystearate, PEG-35 glyceryl triricinoleate, PEG-15 hydroxystearate, and mixtures thereof, the solubilizer layer encapsulated by an outer layer comprising a composition selected from the group consisting of: 1) a composition comprising phospholipids and 70%-79% phosphatidylcholine, 2) a composition comprising 94%-100% phosphatidylcholine, and 3) analogs thereof, all of the foregoing comprising a nanodispersed composition that is mixed with water or an aqueous solution to form a stable aqueous formulation suitable for medical use.

In another embodiment, the aqueous pharmaceutical composition comprises an active substance surrounded by a solubilizer layer comprising at least one polyethylene glycol selected from the group consisting of PEG-40 glyceryl trihydroxystearate, PEG-35 glyceryl triricinoleate, PEG-15 hydroxystearate, and mixtures thereof, wherein the solubilizer layer further comprises a poloxamer selected from the group consisting of poloxamer 188, poloxamer 407, and polyethylene glycol, the solubilizer layer encapsulated by an outer layer comprising a composition selected from the group consisting of: 1) a composition comprising phospholipids and 70%-79% phosphatidylcholine, 2) a composition comprising 94%-100% phosphatidylcholine, and 3) analogs thereof, all of the foregoing comprising a nanodispersed composition that is mixed with water or an aqueous solution to form a stable aqueous formulation suitable for medical use.

Additional aspects of the claimed subject matter will be set forth in part in the description which follows, and in part will be obvious from the description, or may be learned by practice of the claimed subject matter. The aspects of the claimed subject matter will be realized and attained by means of the elements and combinations particularly pointed out in the appended claims. It is to be understood that both the foregoing general description and the following detailed description are exemplary and explanatory only and are not restrictive of the disclosed subject matter, as claimed.

### BRIEF DESCRIPTION OF THE SEVERAL VIEWS OF THE DRAWINGS

The accompanying drawings, which are incorporated in and constitute part of this specification, illustrate embodiments of the claimed subject matter and together with the description, serve to explain the principles of the claimed subject matter. The embodiments illustrated herein are presently preferred, it being understood, however, that the claimed subject matter is not limited to the precise arrangements and instrumentalities shown, wherein:

FIG. 1 is an illustration of an aqueous pharmaceutical composition for enhancing bioavailability of an active substance in a patient, in accordance with one embodiment.

FIG. 2 is a flowchart for a process of making the aqueous pharmaceutical composition for enhancing bioavailability of an active substance in a patient, in accordance with one embodiment.

FIG. 3 is a diagram showing the aqueous pharmaceutical composition for enhancing bioavailability of an active substance in a patient in action, in accordance with one embodiment.

FIG. 4 is an illustration showing different methods of delivery of the aqueous pharmaceutical composition to a patient, according to one embodiment.

### DETAILED DESCRIPTION

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions or modifications may be made to the elements illustrated in the drawings, and the methods described herein may be modified by substituting, reordering, or adding additional stages or components to the disclosed methods and devices. Accordingly, the following detailed description does not limit the disclosed embodiments. Instead, the proper scope of the disclosed embodiments is defined by the appended claims.

The claimed embodiments, overcomes the challenges identified in the prior art by introducing a novel macromolecular delivery system that enhances the bioavailability of active substances, particularly those poorly soluble in water, while effectively evading immune system neutralization and simplifying production processes. The claimed embodiments address the problem of poor solubility and low bioavailability of active pharmaceutical ingredients (APIs) by utilizing specific solubilizers—polyethylene glycols such as PEG-40 glyceryl trihydroxystearate (otherwise known as Kolliphor RH 40 in the industry), PEG-35 glyceryl triricinoleate (otherwise known as Kolliphor EL in the industry), and PEG-15 hydroxystearate (otherwise known as Kolliphor HS 15 in the industry)—to solubilize the active substances and form a nanodispersed composition. This nanoscopic formulation increases the surface area of the active substance, enhancing its solubility and facilitating more efficient absorption into systemic circulation. By improving solubility at the molecular level, the claimed embodiments ensure that a higher percentage of the active substance becomes bioavailable, thereby increasing therapeutic efficacy.

Further, the claimed embodiments overcome the obstacle of immune system neutralization by incorporating phosphatidylcholine to create an “immune trap,” thereby camouflaging the active substance within a macromolecular structure that is biocompatible and less likely to be targeted by the immune system. This allows the active substances to bypass immune detection, increasing the amount that reaches the target site and enhancing therapeutic outcomes. Furthermore, the claimed embodiments minimize production complexity and costs by reducing the need for multiple additional substances and simplifying the manufacturing process by streamlining production by utilizing readily available, pharmaceutically acceptable components and straightforward mixing and solubilization steps. This reduces manufacturing expenses and facilitates easier implementation. Also, the claimed embodiments address toxicity concerns associated with certain solubilizers and surfactants by employing biocompatible and safe materials such as natural phosphatidylcholine.

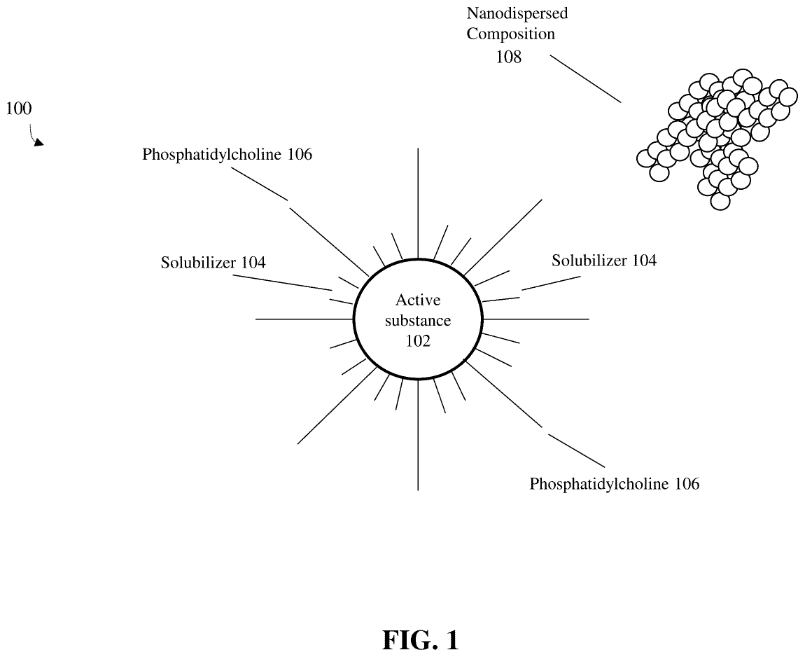

Referring now to the drawing figures in which like reference designators refer to like elements, there is shown in FIG. 1 an illustration of an aqueous pharmaceutical composition 100 for enhancing bioavailability of an active substance 102 in a patient, in accordance with one embodiment. This figure provides a cross-sectional view of a single nanoparticle of the aqueous pharmaceutical composition, highlighting its multi-layered architecture and the specific components that contribute to its function. At the core of the nanoparticle of the aqueous pharmaceutical composition 100 is the active substance 102, represented as the central sphere in the figure. This active substance can be any therapeutic agent, such as a poorly water-soluble molecule, including pharmaceuticals, vitamins, peptides, proteins, or plant extracts. The active substance is encapsulated within the delivery vehicle to protect it from degradation and enhance its solubility and bioavailability.

“Macromolecular” refers to large molecules that are composed of thousands or even millions of atoms. These molecules are typically formed by the polymerization of smaller units called monomers. Macromolecules are fundamental components in both biological and synthetic systems and include substances like proteins, nucleic acids (DNA and RNA), polysaccharides (like starch and cellulose), and synthetic polymers (like plastics and polyethylene glycol).

Surrounding the active substance is the solubilizer layer 104, depicted as a concentric shell encasing the core. This layer consists of solubilizing agents, such as PEG-40 glyceryl trihydroxystearate with a molecular mass of 40 mM, PEG-35 glyceryl triricinoleate with a molecular mass of 35 mM, and PEG-15 hydroxystearate with a molecular mass of 15 mM, which are polyethylene glycol (PEG) derivatives, or any analogs of the foregoing. The solubilizer molecules arrange themselves around the hydrophobic active substance through hydrophobic interactions, forming a micelle-like structure. This configuration enhances the solubility of the active substance by shielding it from the aqueous environment and allowing it to disperse uniformly in water. The solubilizer layer stabilizes the active substance and facilitates its delivery to the target site.

PEG-40 glyceryl trihydroxystearate, otherwise known as PEG-40 hydrogenated castor oil or Kolliphor RH 40, is produced by reacting hydrogenated castor oil with approximately 40 moles of ethylene oxide. This process ethoxylates the castor oil, enhancing its solubility in water while retaining lipophilic (oil-loving) properties. PEG-35 glyceryl triricinoleate, otherwise known as polyethylene glycol 35 castor oil or Kolliphor EL, is a nonionic surfactant and solubilizing agent produced by reacting castor oil with approximately 35 moles of ethylene oxide, a process called ethoxylation. This modification introduces polyethylene glycol chains into the castor oil molecule, enhancing its solubility in water while retaining lipophilic (oil-attracting) properties. PEG-15 hydroxystearate, otherwise known as polyethylene glycol 15 hydroxystearate or Kolliphor HS 15, is a nonionic surfactant and solubilizing agent produced by the ethoxylation of 12-hydroxystearic acid with approximately 15 moles of ethylene oxide. This process results in a molecule that possesses both hydrophilic (water-attracting) and hydrophobic (oil-attracting) properties, making it an effective emulsifier and solubilizer.

In addition to the solubilizing agents described above, mixtures of these polyethylene glycols can be utilized to optimize solubilization. Said mixtures may be used with specific weight ratios for achieving the desired solubility and stability. For example, the weight ratio of PEG-40 glyceryl trihydroxystearate to PEG-35 glyceryl triricinoleate is about 2.0 to 2.5 parts PEG-40 glyceryl trihydroxystearate to 0.5 to 1.0 part PEG-35 glyceryl triricinoleate. This mixture is then combined with PEG-15 hydroxystearate in a weight ratio of 3.0 parts of the PEG-40 glyceryl trihydroxystearate and PEG-35 glyceryl triricinoleate mixture to 1.0 part PEG-15 hydroxystearate.

The solubilizer may also include poloxamers in addition to the PEGs, enhancing the solubilization and stability of the active substance. Poloxamers may include poloxamer 188 (also known as Kolliphor F68), consisting of 81% polyethylene glycol and 19% polypropylene glycol; poloxamer 407 (also known as Kolliphor F127), consisting of 73% polyethylene glycol and 27% polypropylene glycol; and polyethylene glycol (also known as Kolliphor E 400), consisting of 100% polyethylene glycol. These poloxamers are added to the polyethylene glycol solubilizer in a weight ratio of about 0.1 to 1.0 part poloxamer to 1.0 to 4.0 parts polyethylene glycol. The PEGs can be used individually or in mixtures as described above. In terms of general proportions, the weight ratio of the active substance to the solubilizer typically ranges from 1.0:1.0 to 1.0:60.0, depending on the solubility requirements of the active substance. When phosphatidylcholine is included to form the immune trap, its weight ratio to the solubilizer-active substance mixture ranges from 1.0:1.0 to 1.0:50.0.

Poloxamer 188 and 407 are nonionic, triblock copolymer surfactants widely used in pharmaceutical, cosmetic, and biomedical applications for its solubilizing, stabilizing, and emulsifying properties. Polyethylene glycol is a polyether compound derived from petroleum with many applications, from industrial manufacturing to medicine.

Encapsulating the solubilizer layer 104 is the phosphatidylcholine outer layer 106, referred to as the immune trap. This layer is composed of phosphatidylcholine molecules, such as a composition comprising phospholipids and 70%-79% phosphatidylcholine (otherwise known as Lipoid S80 in the industry) or a composition comprising 94%-100% phosphatidylcholine (Lipoid S100) or analogs thereof. Phosphatidylcholine is a natural phospholipid with amphiphilic properties, featuring hydrophilic heads and hydrophobic tails. In the delivery system 100, these molecules orient themselves with their hydrophobic tails interacting with the solubilizer layer and their hydrophilic heads facing outward toward the aqueous environment.

Phosphatidylcholine is a phospholipid, a type of lipid molecule that is a major component of cell membranes in the body. It consists of a glycerol backbone attached to two fatty acid chains and a phosphate group linked to a choline molecule. This structure gives phosphatidylcholine both hydrophilic (water-attracting) and hydrophobic (water-repelling) properties, allowing it to form the lipid bilayers that make up cell membranes. Phosphatidylcholine is derived from soybean lecithin and is commonly used as an emulsifier, liposome-forming agent, and solubilizer.

The outer layer 106 serves multiple functions, such as immune invasion, biocompatibility and stability. With reference to immune invasion, the phosphatidylcholine layer masks the underlying components of the delivery system from recognition by macrophages and other immune cells, effectively creating an immune trap. This prevents the premature neutralization of the active substance, allowing for increased bioavailability and therapeutic efficacy. With reference to biocompatibility, being a component of natural cell membranes, phosphatidylcholine enhances the biocompatibility of the delivery system, reducing the risk of adverse reactions. With reference to stability, the outer layer contributes to the structural integrity and stability of the nanoparticle in physiological conditions The entire assembly forms a nanodispersed composition 108, represented by multiple nanoparticles uniformly distributed in the medium. The nanoparticles are of nanoscale dimensions, typically ranging from 10 to 100 nanometers in diameter. The nanoscale size offers several advantages, such as enhanced absorption. Small particle size increases the surface area-to-volume ratio, facilitating better interaction with biological membranes and improving absorption rates. The nanoscale size also facilitates improved penetration. Nanoparticles can penetrate biological barriers more effectively than larger particles, allowing for targeted delivery to specific tissues or cells. The nanoscale size also provides uniform distribution. The nanodispersed nature ensures consistent dosing and homogeneity in the formulation, which is critical for reproducible therapeutic outcomes. The described macromolecular delivery system 100 effectively overcomes the limitations of poor water solubility and immune system neutralization that are common with traditional drug delivery methods.

A nanodispersed composition refers to a formulation in which particles of one or more substances are dispersed uniformly throughout a medium at the nanometer scale-that is, particles typically ranging in size from 1 to 100 nanometers (nm). In such compositions, the active substances are present as nanoparticles, which significantly alters their physical, chemical, and biological properties compared to larger-sized particles.

Nanodispersed compositions have several key characteristics. The nanoparticles have extremely small dimensions, leading to a high surface area-to-volume ratio, which enhances interactions with biological systems and can improve solubility and reactivity. The uniform dispersion of nanoparticles within the medium prevents aggregation and ensures consistent dosing and efficacy. When poorly water-soluble substances are reduced to nanoscale particles, they exhibit increased solubility and faster dissolution rates, thereby improving their bioavailability. Additionally, nanoparticles can more easily cross biological barriers, such as cell membranes and the gastrointestinal lining, enhancing absorption and therapeutic effects. Nanodispersed systems can also be engineered to release active substances at controlled rates, improving treatment outcomes and reducing side effects.

The macromolecular delivery system utilizes a nanodispersed composition resulting from the combination of the active substance, solubilizer, and phosphatidylcholine. The assembly of the layers in FIG. 1 results in nanoparticles that are uniformly dispersed in the aqueous medium, creating the nanodispersed composition. This offers several advantages. The small size allows for better penetration through biological membranes, leading to enhanced absorption and increased efficacy. The nanoparticles protect the active substance from degradation due to environmental factors or metabolic processes, improving stability. The nanodispersed composition can facilitate targeted delivery to specific tissues or cells, improving therapeutic outcomes. Enhanced bioavailability can allow for lower doses of the active substance to achieve the desired therapeutic effect. Moreover, the versatility of nanodispersed compositions means they can be adapted for various routes of administration, including oral, topical, and transmucosal delivery With regard to the immune trap, phosphatidylcholine enables nanoparticles in the macromolecular delivery system to evade immune detection at the molecular level by mimicking the natural components of cell membranes, thereby presenting a “self” signal to the immune system. Phosphatidylcholine is a major phospholipid found in biological membranes, composed of a hydrophilic (water-attracting) head and two hydrophobic (water-repelling) fatty acid tails. When phosphatidylcholine molecules form the outer layer of the nanoparticle, they arrange themselves so that the hydrophobic tails interact with the underlying solubilizer layer, while the hydrophilic heads face outward toward the aqueous environment. This orientation creates a surface that closely resembles the external leaflet of natural cell membranes.

At the molecular level, immune cells like macrophages recognize and eliminate foreign particles through receptors that detect specific patterns absent in host cells. By coating the nanoparticle with phosphatidylcholine, the delivery system effectively camouflages itself with a biocompatible surface that is familiar to the immune system. This similarity reduces the likelihood of the nanoparticle being identified as foreign and targeted for phagocytosis. Additionally, the phosphatidylcholine layer inhibits the adsorption of opsonins-proteins in the blood that bind to foreign particles and mark them for immune clearance. By preventing opsonin binding, the nanoparticles avoid being tagged for destruction by macrophages.

The hydrophilic head groups of phosphatidylcholine interact with water molecules to form a hydration shell around the nanoparticle. This shell serves as a physical barrier, further reducing protein adsorption and recognition by immune cells. The overall effect is a stealth-like property that allows the nanoparticles to circulate in the bloodstream for extended periods without eliciting an immune response. This prolonged circulation increases the chances of the nanoparticles reaching their target sites, thereby enhancing the bioavailability and therapeutic efficacy of the active substance they carry.

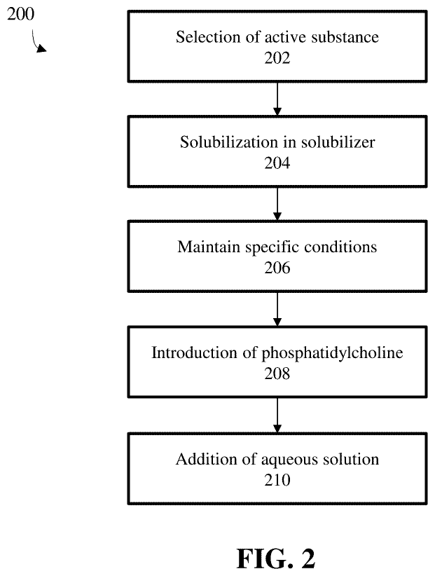

FIG. 2 is a flowchart for a process 200 of making the aqueous pharmaceutical composition for enhancing bioavailability of an active substance in a patient, in accordance with one embodiment. The process begins with the step 202, which involves selection of the desired active substance or substances. These active substances are typically poorly water-soluble molecules that require enhanced bioavailability for effective therapeutic action. Examples include pharmaceuticals, vitamins, peptides, proteins, lipids, and plant extracts. In one embodiment, the active substance comprises a poorly water-soluble molecule selected from the group consisting of vitamins, peptides, proteins, lipids, plant extracts, and pharmaceutical agents. In another embodiment, the active substance is selected from the group consisting of insulin, proinsulin, stem cells, peptides, interferons, chondroitin, methylsulfonylmethane (MSM), glucosamine, beta-carotene, alpha-lipoic acid, ursodeoxycholic acid, folic acid, lecithin, collagen, peptides, exosomes, sildenafil, vitamin E, orlistat, carnitine, fish oil, plant extracts and proteins. The chosen active substance will form the core of the nanodispersed composition.

In step 204, the selected active substance is solubilized in a solubilizer that has been preheated to approximately 60° C. The solubilizers used are polyethylene glycol (PEG) derivatives such as PEG-40 glyceryl trihydroxystearate, PEG-35 glyceryl triricinoleate, or PEG-15 hydroxystearate. The active substance is thoroughly mixed with the solubilizer under controlled temperature conditions to form a homogeneous mixture. The weight ratio of the active substance to the solubilizer is carefully measured, typically ranging from 1.0:1.0 to 1.0:60.0, depending on the solubility requirements of the active substance.

Following solubilization, in step 206, the mixture undergoes further processing to form a nanodispersed composition. This involves maintaining the mixture under specific conditions to ensure the formation of nanoparticles, with the active substance encapsulated within the solubilizer matrix. The nanoparticles typically range in size from 10 to 100 nanometers, providing a large surface area that enhances interaction with biological systems and improves solubility and absorption.

In step 208, phosphatidylcholine-such as a composition comprising phospholipids and 70%-79% phosphatidylcholine, a composition comprising 94%-100% phosphatidylcholine, and analogs thereof-is introduced into the nanodispersed composition. The phosphatidylcholine integrates into the molecular associate formed by the solubilizer and active substance. This step is critical as it creates the “immune trap,” which helps the delivery system evade recognition and neutralization by macrophages and other immune cells. The weight ratio of phosphatidylcholine to the associate is adjusted, typically ranging from 1.0:1.0 to 1.0:50.0, to achieve optimal integration and functionality.

Next, in step 210, the mixture is cooled to approximately 30° C. and an aqueous solution is added. Cooling stabilizes the nanodispersed composition and prepares it for the addition of water or an aqueous solution. The aqueous solution may contain preservatives (such as parabens or sorbic acid), antioxidants, or water-soluble vitamins like vitamin C. Adding water or the aqueous solution transforms the mixture into a transparent, stable aqueous formulation suitable for administration. The components are mixed thoroughly to ensure uniform dispersion and stability of the final product. Finally, the macromolecular delivery system is formed. The outcome is a stable aqueous formulation containing the nanodispersed active substance encapsulated within the solubilizer and phosphatidylcholine layers, and is suitable for various routes of administration, including oral, topical, and transmucosal delivery, as well as intravenous and intramuscular.

The aqueous solution in the macromolecular delivery system serves as the medium in which the nanodispersed composition of the active substance is dispersed to form a stable, bioavailable formulation suitable for administration. In one embodiment, the aqueous solution includes purified water as the primary solvent, providing a biocompatible medium for the dispersion of the nanodispersed particles. To ensure stability and shelf-life, preservatives such as parabens (e.g., methylparaben, ethylparaben) or sorbic acid may be included to inhibit microbial growth. Water-soluble vitamins, such as vitamin C or those from the B-complex group, may be added in weight ratios to water ranging from 1.0:20.0 to 1.0:1000.0, acting as antioxidants and providing additional therapeutic benefits. Optional antioxidants like tocopherols (vitamin E) may be included to prevent oxidation of sensitive components within the solution. The pH may be adjusted to match physiological conditions, typically between pH 4 and 8, to enhance stability and compatibility.

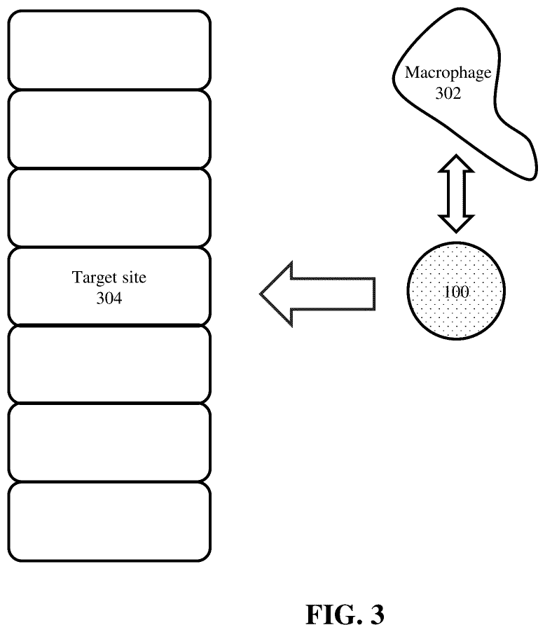

FIG. 3 is a diagram showing the aqueous pharmaceutical composition for enhancing bioavailability of an active substance in a patient in action, in accordance with one embodiment. This figure provides a visual representation of the interaction between the pharmaceutical composition particle 100 and immune cells, highlighting the role of the phosphatidylcholine layer in preventing immune recognition. A macrophage 302 is depicted as part of the body's immune surveillance system. A macrophage attempts to recognize and engulf a foreign particle through phagocytosis. Macrophages play a critical role in identifying and neutralizing pathogens and foreign substances by recognizing specific surface markers or the absence of “self” signals on particles.

Enveloping the solubilizer-coated active substance is an outer layer of phosphatidylcholine, which acts as an immune trap. The phosphatidylcholine layer mimics the natural phospholipid components of cell membranes, presenting a “self” signal to the immune system. This camouflage prevents macrophages from recognizing the particles as foreign, thereby avoiding phagocytosis. In the figure, the macrophage is shown passing by the pharmaceutical composition particle 100 without initiating an immune response.

The pharmaceutical composition particle 100 is shown traveling through the bloodstream or extracellular matrix, unimpeded by immune cells, toward the target site 304. Upon reaching the vicinity of the target cells, the nanoparticle interacts with cell membranes. Due to its nanoscale size and the presence of the phosphatidylcholine layer, the particle facilitates cellular uptake through mechanisms such as endocytosis or membrane fusion. Once internalized, the delivery system releases the active substance within or near the target cells, allowing it to exert its therapeutic effect. The figure illustrates the successful delivery and action of the active substance at the target site, emphasizing the efficiency of the delivery system.

The mechanism depicted addresses a significant limitation in prior drug delivery methods-the premature clearance of therapeutic agents by the immune system. By incorporating an immune trap using phosphatidylcholine, the delivery system prolongs the circulation time of the active substance, increases its bioavailability, and improves treatment outcomes. This strategy reduces the required dosage and minimizes potential side effects associated with higher concentrations of active substances.

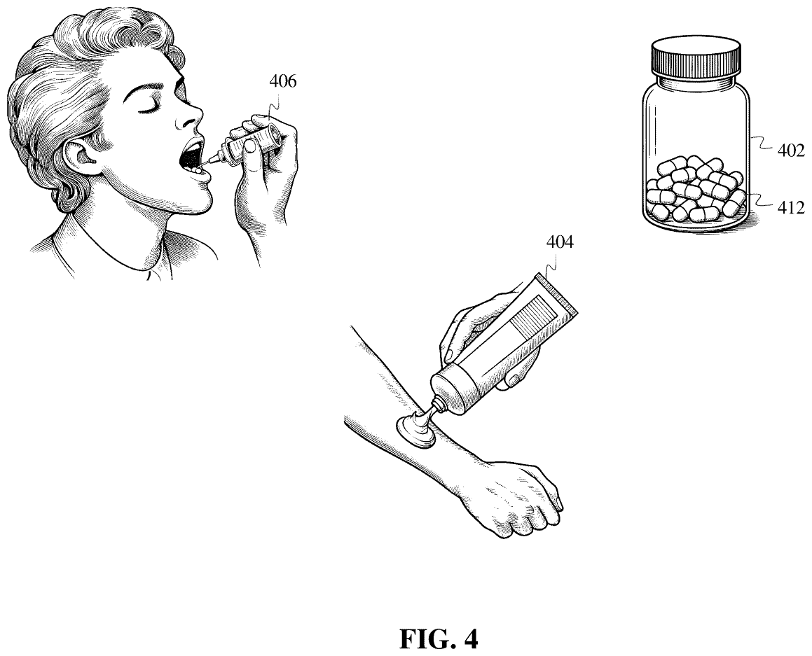

FIG. 4 is an illustration showing different methods of delivery of the aqueous pharmaceutical composition for enhancing bioavailability of an active substance to a patient, according to one embodiment. One method of administering the aqueous pharmaceutical composition to a patient is via the transmucosal route. Specifically, the aqueous pharmaceutical composition, in liquid form, is presented in a dispenser 406 that inserted into the patient's oral cavity, targeting the mucous membranes. This method involves delivering the active substance through mucosal tissues such as the sublingual (under the tongue) or buccal (inner cheek) areas. The transmucosal delivery route facilitates rapid absorption of the active substance directly into systemic circulation, bypassing the gastrointestinal tract and first-pass hepatic metabolism. This enhances bioavailability and allows for quick therapeutic effects, which is particularly beneficial for active substances requiring prompt onset of action.

Another method of administering the aqueous pharmaceutical composition to a patient is via topical application. In this method, a cream or lotion 404 containing the active substance encapsulated within the macromolecular delivery system may be applied to the skin on the arm, for example. The topical formulation is designed to deliver the active substance transdermally, penetrating the skin barrier to reach underlying tissues or systemic circulation. This method is advantageous for both localized treatment of skin conditions and systemic delivery of active substances without the need for invasive procedures. This method further offers a non-invasive alternative to injections.

Another method of administering the aqueous pharmaceutical composition to a patient is via oral administration. A pill bottle 402 containing capsules or tablets 412 is shown, representing the formulation of the aqueous pharmaceutical composition within an oral dosage form. The macromolecular delivery system enhances the solubility and stability of poorly water-soluble active substances, facilitating their absorption in the gastrointestinal tract. By improving dissolution and protecting the active substance from degradation in the harsh gastric environment, the system increases bioavailability and therapeutic efficacy. Oral administration offers a convenient and non-invasive route, promoting patient compliance and ease of use in both clinical and home settings.

While certain embodiments have been described, other embodiments may exist. Further, the disclosed methods'stages may be modified in any manner, including by reordering stages and/or inserting or deleting stages, without departing from the claimed subject matter. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. An aqueous pharmaceutical composition for enhancing bioavailability of an active substance in a patient, comprising:
an active substance completely surrounded by a solubilizer layer comprising at least one polyethylene glycol selected from the group consisting of PEG-40 glyceryl trihydroxystearate, PEG-35 glyceryl triricinoleate, PEG-15 hydroxystearate, and mixtures thereof, the solubilizer layer defining an internal hydrophobic phase enclosing the active substance and an external hydrophilic surface in contact with an aqueous medium;
the solubilizer layer completely encapsulated by an outer layer comprising a composition selected from the group consisting of: 1) a composition comprising phospholipids and 70%-79% phosphatidylcholine, and 2) a composition comprising 94%-100% phosphatidylcholine, wherein said outer layer is cholesterol-free;
all of the foregoing comprising a nanodispersed composition that is mixed with water or an aqueous solution to form a stable aqueous formulation suitable for medical use.

2. The aqueous pharmaceutical composition of claim 1, wherein the active substance comprises a poorly water-soluble molecule selected from the group consisting of vitamins, peptides, proteins, lipids, plant extracts, and pharmaceutical agents.

3. The aqueous pharmaceutical composition of claim 1, wherein the active substance is selected from the group consisting of insulin, proinsulin, stem cells, peptides, interferons, chondroitin, methylsulfonylmethane (MSM), glucosamine, beta-carotene, alpha-lipoic acid, ursodeoxycholic acid, folic acid, lecithin, collagen, peptides, exosomes, sildenafil, vitamin E, orlistat, carnitine, fish oil, plant extracts and proteins.

4. The aqueous pharmaceutical composition of claim 1, wherein the solubilizer further comprises a poloxamer selected from the group consisting of poloxamer 188, poloxamer 407 and polyethylene glycol, added to the polyethylene glycol solubilizer.

5. The aqueous pharmaceutical composition of claim 4, wherein the aqueous solution comprises at least one of the following: preservatives, antioxidants or water-soluble vitamin C.

6. The aqueous pharmaceutical composition of claim 5, wherein the preservative is selected from the group consisting of: parabens, and sorbic acid.

7. The aqueous pharmaceutical composition of claim 6, wherein the stable aqueous formulation is adapted for transmucosal delivery through mucous membranes.

8. The aqueous pharmaceutical composition of claim 6, wherein the stable aqueous formulation is formulated for topical application to the skin.

9. The aqueous pharmaceutical composition of claim 6, wherein the stable aqueous formulation is adapted for oral administration.

10. The aqueous pharmaceutical composition of claim 6, wherein the stable aqueous formulation is adapted for oral administration via formation into pill form.

11. An aqueous pharmaceutical composition for enhancing bioavailability of an active substance in a patient, comprising:
an active substance completely surrounded by a solubilizer layer comprising at least one polyethylene glycol selected from the group consisting of PEG-40 glyceryl trihydroxystearate, PEG-35 glyceryl triricinoleate, PEG-15 hydroxystearate, and mixtures thereof, the solubilizer layer defining an internal hydrophobic phase enclosing the active substance and an external hydrophilic surface in contact with an aqueous medium, wherein the solubilizer layer further comprises a poloxamer selected from the group consisting of poloxamer 188, poloxamer 407, and polyethylene glycol,
the solubilizer layer completely encapsulated by an outer layer comprising a composition selected from the group consisting of: 1) a composition comprising phospholipids and 70%-79% phosphatidylcholine, and 2) a composition comprising 94%-100% phosphatidylcholine, wherein said outer layer is cholesterol-free;
all of the foregoing comprising a nanodispersed composition that is mixed with water or an aqueous solution to form a stable aqueous formulation suitable for medical use.

12. The aqueous pharmaceutical composition of claim 11, wherein the active substance comprises a poorly water-soluble molecule selected from the group consisting of vitamins, peptides, proteins, lipids, plant extracts, and pharmaceutical agents.

13. The aqueous pharmaceutical composition of claim 11, wherein the active substance is selected from the group consisting of insulin, proinsulin, stem cells, peptides, interferons, chondroitin, methylsulfonylmethane (MSM), glucosamine, beta-carotene, alpha-lipoic acid, ursodeoxycholic acid, folic acid, lecithin, collagen, peptides, exosomes, sildenafil, vitamin E, orlistat, carnitine, fish oil, plant extracts and proteins.

14. (canceled)

15. The aqueous pharmaceutical composition of claim 13, wherein the aqueous solution comprises at least one of the following: preservatives, antioxidants or water-soluble vitamin C.

16. The aqueous pharmaceutical composition of claim 15, wherein the preservative is selected from the group consisting of: parabens, and sorbic acid.

17. The aqueous pharmaceutical composition of claim 16, wherein the stable aqueous formulation is adapted for transmucosal delivery through mucous membranes.

18. The aqueous pharmaceutical composition of claim 16, wherein the stable aqueous formulation is formulated for topical application to the skin.

19. The aqueous pharmaceutical composition of claim 16, wherein the stable aqueous formulation is adapted for oral administration.

20. The aqueous pharmaceutical composition of claim 16, wherein the stable aqueous formulation is adapted for oral administration via formation into pill form.
